(19) United States
(12) Patent Application Publication
(10) Pub. No.: US 2020/0061342 A1
(43) Pub. Date: Feb. 27, 2020

(54) Catheter Pouch

(71) Applicant: Patricia L. Chavis, Pembroke, NC (US)

(72) Inventor: Patricia L. Chavis, Pembroke, NC (US)

(21) Appl. No.: 16/543,866

(22) Filed: Aug. 19, 2019

Related U.S. Application Data
(60) Provisional application No. 62/721,709, filed on Aug. 23, 2018.

(51) Int. Cl. A61M 25/02; A61M 1/28

(52) U.S. Cl. CPC A61M 25/02; A61M 2025/0206; A61M 1/285

(57) ABSTRACT

A catheter pouch. The catheter pouch has a strap with a first fastener disposed on opposing ends of a strap, such that engagement of the first fastener removably attaches a first end of the strap to a second end of the strap. A pouch is attached to the strap. The interior volume of the pouch is accessible via an opening. The opening has a second fastener, operation of which allows for an open and a closed configuration of the pouch. At least one hook is also disposed on the pouch, which can be utilized to removably secure the pouch to a garment. The catheter pouch allows a user to comfortably and discretely support and store a catheter.

### CROSS REFERENCE TO RELATED APPLICATIONS

This application claims the benefit of U.S. Provisional Application No. 62/721,709 filed on Aug. 23, 2018. The above identified patent application is herein incorporated by reference in its entirety to provide continuity of disclosure.

### BACKGROUND OF THE INVENTION

The present invention relates to catheter storage devices. More particularly, the present invention provides for a wearable catheter pouch that can be utilized to conveniently store and conceal a catheter.

Many people with reduced kidney function seek medical treatment. One such form of treatment is through peritoneal dialysis where a small tube known as a catheter is threaded through the abdomen of the patient and into the peritoneum. One end of the catheter remains outside of the patient's body and is utilized to pass a fluid through the catheter and into the peritoneum in order to clean the patient's blood inside their body. The catheter is secured in place and covered by a sterile dressing over the insertion point. As the catheter is left in place for extended periods of time, long-term care must be adhered to in order to prevent complications and infection.

A length of the catheter that resides outside of the patient's body is utilized to instill or drain a peritoneal dialysis solution. When not so utilized, the end of this portion of the catheter is capped off. This length of the catheter that remains outside of the patient's body is free to hang, and if unsecured can become entangled in clothing or snag on objects the patient may brush against. Such interactions with clothing and objects may damage the catheter, or the insertion site, which may require a trained medical professional to repair and/or replace the catheter or treat the insertion site.

Devices have been disclosed in the known art that relate to catheter storage devices that attempt to address these issues. These include devices that have been patented and disclosed in patent application publications. However, these devices have several drawbacks of their own. Some of these devices secure the catheter to the patient themselves, resulting in reduced mobility of the patient, and discomfort. Some devices allow for mobility of the patient, but do not adequately conceal the catheter. The patient may be embarrassed where the catheter is visible, either hanging outside of the clothes, or showing through the clothes as a bulky mass. Accordingly, a device that securely stores, while concealing, the catheter is desired. The present invention substantially diverges in design elements from the known art and consequently it is clear that there is a need in the art for an improvement to existing catheter storage devices. In this regard the present invention substantially fulfills these needs.

### SUMMARY OF THE INVENTION

In view of the foregoing disadvantages inherent in the known types of catheter storage devices now present n the prior art, the present invention provides a device that securely stores, while concealing, a catheter. The present catheter pouch comprises a strap with a fastener disposed on opposing ends of the strap and a pouch attached to the strap that can be selectively opened or closed which stores a catheter.

Other objects, features and advantages of the present invention will become apparent from the following detailed description taken in conjunction with the accompanying drawings.

### BRIEF DESCRIPTION OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a perspective view of an embodiment of the catheter pouch.

FIG. 2 shows a top-down view of an embodiment of the catheter pouch.

FIG. 3 shows a perspective view of an embodiment of the catheter pouch in use.

### DETAILED DESCRIPTION OF THE INVENTION

Reference is made herein to the attached drawings. Like reference numerals are used throughout the drawings to depict like or similar elements of the catheter pouch. For the purposes of presenting a brief and clear description of the present invention, a preferred embodiment will be discussed as used for the catheter pouch. The figures are intended for representative purposes only and should not be considered to be limiting in any respect.

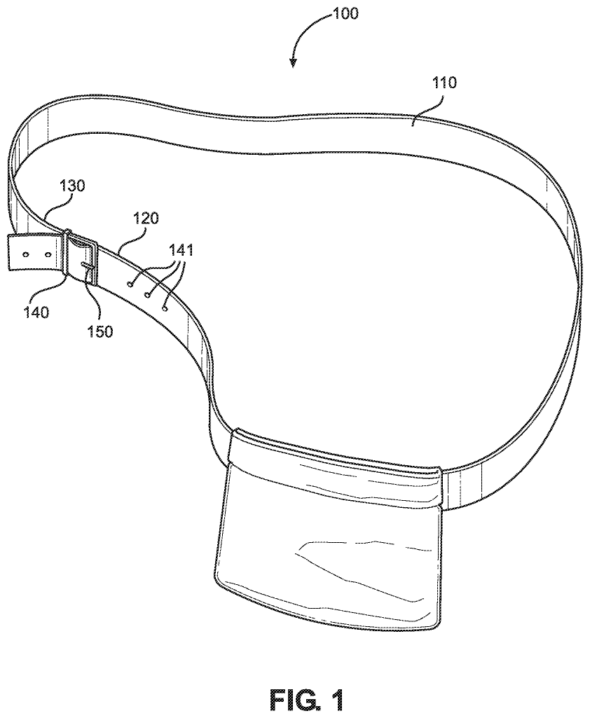

Referring now to FIG. 1, there is shown a perspective view of an embodiment of the catheter pouch. The catheter pouch 100 comprises a strap 110 with a first end 120 disposed opposite a second end 130. A first fastener 140 is disposed on the first end 120 and the second end 130, such that engagement of the first fastener 140 removably attaches the first end 120 to the second end 130. In one embodiment, the first fastener 140 further comprises a receiving loop end disposed on the first end 120 and a complementary hook disposed on the second end 130. A user is able to secure the strap 110 around their waist by passing the complementary hook into the receiving loop. In a further embodiment, the first fastener 140 is a hook and bar closure. One of ordinary skill in the art will understand that various types of fasteners and closures are able to be used, and will vary depending on the size, shape, and aesthetics desired. In the shown embodiment, the strap 110 is comprised of an elastic material. Use of an elastic material provides added comfort when securing the strap 110 around the waist of the individual as the elastic material provides a snug, but not uncomfortably tight fit, around said waist. In other embodiments, a plurality of apertures 141 is disposed along the length of the strap 110 and the first fastener 140 comprises a belt buckle.

In the shown embodiment the first fastener 140 comprises an adjuster 150 that enables an individual to selectively adjust a length of the strap. In various embodiments, the length of the strap is of sufficient length to encircle individuals of various sizes and shapes. The selective adjustment of the length of the strap 110 allows an individual to determine how tightly the strap is secured around the individual's waist. The selective adjustment of the tightness of the strap also enables a user to snugly adjust the strap over the catheter without applying so much pressure that the strap deforms the catheter.

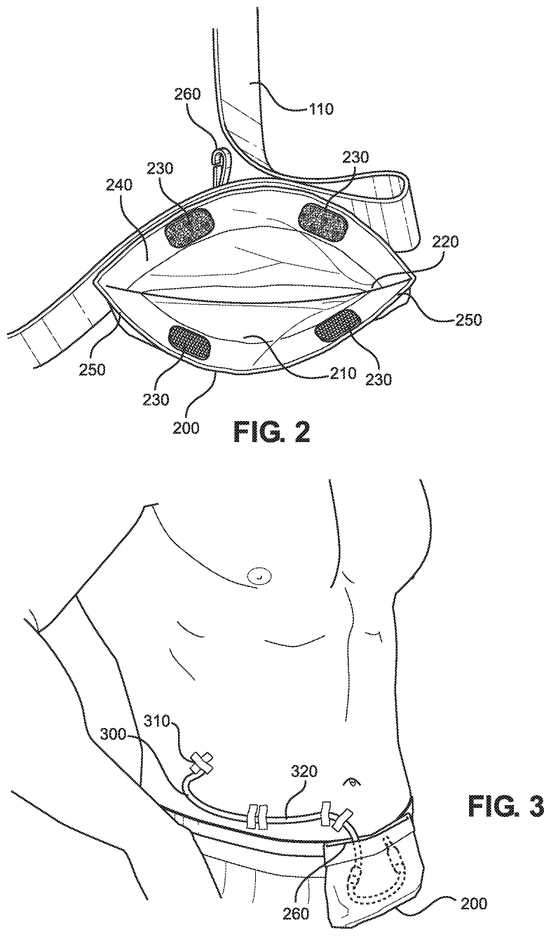

Referring now to FIG. 2, there is shown a top-down view of an embodiment of the catheter pouch. A pouch 200 is disposed on the strap. In the shown embodiment, the pouch 200 is closed on three sides, and the fourth side is open. The pouch 200 defines an interior volume 210, wherein the interior volume 210 is accessible via an opening 220 of the pouch 200. In the shown embodiment, the opening 220 is the fourth open side of the pouch. In one embodiment, the pouch 200 is removably securable to the strap 110. In such an embodiment, an individual removes the pouch 200 from the strap 110 for easier access to the interior volume 210, and for easier cleaning of the pouch 200 between uses.

The opening 220 of the pouch 200 has a second fastener 230. The second fastener 230 allows for the opening 220 of the pouch 200 to be moveable between an open configuration, allowing an individual to access the interior volume 210, and a closed configuration. The closed configuration is a configuration that still allows a catheter to be threaded through the opening 220. In the shown embodiment, the second fastener 230 is two sets of complimentary hook and loops fasteners disposed on the top surface 240 of the opening 220. The two sets of complimentary hook and loop fasteners are spaced far enough apart such that the catheter is able to be threaded between the edge 250 of the opening 220 and the hook and loop fasteners, or between the two sets of hook and loop fasteners.

As the catheter pouch is configured to rest securely against the individual's body, and the interior volume of the pouch 200 is sized to receive the catheter 300, the catheter is concealed from view. In one embodiment the pouch 200 further comprises at least one hook 260. The at least one hook 260 is utilized to further secure the catheter pouch to an article of clothing. In this manner the catheter pouch is secured to an individual's underwear or other article of clothing to further conceal the presence of the catheter 300.

Referring now to FIG. 3, there is shown a perspective view of an embodiment of the catheter pouch in use. In the shown embodiment the catheter 300 is secured to an individual's torso via tape near the insertion site 310. A length 320 of the catheter 300 remains outside of the individual's body. This length 320 is threaded through the opening 260 of the pouch 200. The second fastener is secured such that the catheter pouch is in a closed configuration, while allowing the length 320 of the catheter 300 to be threaded in through the opening 260 and stored in the interior volume of the pouch 200. In such a manner a catheter 300 is securely stored in the catheter pouch. In various embodiments, the opening 260 of the pouch 200, when the pouch is in a closed configuration, is sized to correspond to the diameter of the catheter 300. In this manner, a seal is formed between the pouch 200 and the catheter 300 to prevent undesirable materials from entering the pouch 200. In further embodiments, a cushioning material is disposed around a perimeter of the pouch 200 in order to ensure a snug fit of the pouch 200 around a circumference of the catheter 300.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) A catheter pouch, comprising:
a strap with a first end disposed opposite a second end;
a first fastener disposed on the first end and the second end, such that engagement of the first fastener removably attaches the first end to the second end;
a pouch disposed on the strap;
the pouch defining an interior volume;
the interior volume accessible via an opening of the pouch;
the opening having a second fastener;
wherein the second fastener allows for the opening to be moveable between an open and a closed configuration;
at least one hook disposed on the pouch.

2) The catheter pouch of claim 1, wherein the strap is comprised of an elastic material.

3) The catheter pouch of claim 1, further comprising a plurality of apertures disposed along a length of the strap.

4) The catheter pouch of claim 3, wherein the first fastener is a belt buckle.

5) The catheter pouch of claim 1, wherein the pouch is removably securable to the strap.

6) The catheter pouch of claim 1, wherein a length of the strap is sized to encircle a waist of a user.

7) The catheter pouch of claim 1, wherein a length of the strap is adjustable.

8) The catheter pouch of claim 1, wherein the opening of the pouch is sized to correspond to a diameter of the catheter.
